(19) United States
(12) Patent Application Publication
(10) Pub. No.: US 2017/0333681 A1
(43) Pub. Date: Nov. 23, 2017

(54) Devices for Assisting with Advancement of Catheters and Related Systems and Methods

(71) Applicant: QXMedical, Inc., Roseville, MN (US)

(72) Inventors: Fernando Di Caprio, St. Paul, MN (US); Gianfranco Panarello, Montreal (CA)

(21) Appl. No.: 15/499,194

(22) Filed: Apr. 27, 2017

Related U.S. Application Data
(60) Provisional application No. 62/328,239, filed on Apr. 27, 2016.

(51) Int. Cl. A61M 25/06; A61M 25/00; A61B 17/34; A61M 25/10

(52) U.S. Cl. CPC A61M 25/0662; A61M 25/0054; A61M 25/0041; A61B 17/3478; A61M 25/10

(57) ABSTRACT

The various embodiments herein relate a catheter advancement device having an elongate shaft and a capsule attached to or integral with the shaft, and related methods for assisting with advancement of catheters such as guiding catheters while reducing damage to the inner wall of the blood vessel. Some capsules have a distal plug portion and a neck portion having a smaller diameter than the plug. Other capsules have a channel defined along the outer surface of the capsule.

### CROSS-REFERENCE TO RELATED APPLICATION(S)

This application claims the benefit under 35 U.S.C. §119(e) to U.S. Provisional Application 62/328,239, filed Apr. 27, 2016 and entitled “Devices for Assisting with Advancement of Guiding Catheters and Related Systems and Methods,” which is hereby incorporated herein by reference in its entirety.

### FIELD OF THE INVENTION

The various embodiments herein relate to advancement devices for assisting with advancement of a catheter through a blood vessel while reducing the risk of damaging the inner wall of the blood vessel with the distal end of the catheter.

### BACKGROUND OF THE INVENTION

A guiding catheter (or “sheath”) is a standard catheter that is generally a long tube with a pre-determined shape. It is typically used to gain access to the vasculature—such as a coronary artery—by advancing the catheter through the access point during an interventional procedure. The pre-determined, typically curved shape of the catheter facilitates accessing a specific branch or other portion of the vasculature that requires such a curvature in the catheter.

One disadvantage of a pre-shaped, curved distal end of a catheter (including, but not limited to, a guiding catheter or sheath) is that the advancement of the catheter can be impeded by the distal end contacting and damaging the inner wall of the vessel through which the catheter is being advanced. For example, as depicted in FIG. 1A, the pre-shaped distal end 12 of the guiding catheter 10 can potentially contact the inner wall 16 of the blood vessel 14 (at the area A as shown) as the catheter 10 is advanced distally through the vessel 14, thereby scraping or otherwise causing damage to the inner wall 16. This scraping (or other damage) of the inner wall of the blood vessel by the catheter distal end 12 is sometimes called a “razor effect.”

As best shown in FIG. 1B, one standard, known technique for overcoming the “razor effect” has been the use of a balloon catheter, such as the balloon catheter 20 as shown in the figure. For example, in use, the balloon catheter 20 can be advanced through the inner lumen 24 of the guiding catheter 10 (or other type of catheter or sheath), positioned such that the balloon 22 is protruding from the distal end of the catheter 10 such that a portion of the balloon 22 is positioned within the distal end of the catheter 10 and a portion extends out of the distal end of the catheter 10, and then the balloon 22 is inflated. Once the balloon 22 is inflated, the guiding catheter 10 can be advanced through the blood vessel 14 with the balloon 22 positioned to prevent direct contact between the distal end of the catheter 10 and the inner wall 16 of the vessel 14, thereby preventing the “razor effect.” Once the catheter 10 is advanced to the desired position, the balloon 22 is deflated and the balloon catheter 20 is withdrawn from the guiding catheter 10 so that the guiding catheter 10 is ready for use.

One disadvantage of the use of a balloon catheter (such as catheter 20 discussed above) to prevent the razor effect is the cost: balloon catheters are expensive. Another disadvantage is that the positioning of a balloon catheter at the distal end of the guiding catheter makes it difficult to inject any contrast or other fluid through the guiding catheter and past the inflated balloon of the balloon catheter. That is, the balloon must be deflated in order to allow for injection and then re-inflated.

Thus, there is a need in the art for an improved method and device for advancing a guiding catheter.

### BRIEF SUMMARY OF THE INVENTION

Discussed herein are various catheter insertion or advancement devices for use in assisting with advancement of a catheter through a blood vessel while reducing damage to the inner wall of the blood vessel.

In Example 1, a catheter advancement assistance device comprises an elongate shaft and a capsule fixedly attached to a distal end of the elongate shaft. The capsule comprises a guidewire lumen defined through the capsule, and an outer diameter substantially similar to an inner diameter of a catheter such that the capsule is sized to be positionable through the catheter.

Example 2 relates to the device according to Example 1, wherein the capsule further comprises a distal portion and a neck extending proximally from the distal portion, wherein the neck has a smaller diameter than the distal portion.

Example 3 relates to the device according to Example 1, wherein the guidewire lumen has an inner diameter that is larger than an outer diameter of a standard guidewire.

Example 4 relates to the device according to Example 1, wherein the guidewire lumen is sized to allow fluid to flow through the lumen when a standard guidewire is positioned therein.

Example 5 relates to the device according to Example 1, wherein the capsule further comprises a channel defined longitudinally along an outer surface of the capsule.

Example 6 relates to the device according to Example 1, wherein the capsule further comprises a lip formed around at least a portion of an outer circumference of the capsule.

Example 7 relates to the device according to Example 6, wherein the lip comprises at least two lip segments formed around the outer circumference of the capsule.

Example 8 relates to the device according to Example 1, wherein the capsule further comprises an expanded distal section, wherein the expanded distal segment is substantially elastic, a non-expanded proximal section having a smaller diameter than the expanded distal section, and a lip formed at a juncture between the expanded distal section and the non-expanded proximal section, wherein the lip is formed around at least a portion of a circumference of the capsule.

Example 9 relates to the device according to Example 1, wherein the capsule further comprises a substantially elastic ridge formed around at least a portion of an outer circumference of the capsule.

Example 10 relates to the device according to Example 9, wherein the substantially elastic ridge comprises at least two substantially elastic rig segments formed around the outer circumference of the capsule.

Example 11 relates to the device according to Example 1, wherein the capsule further comprises a slot defined in a distal end of the capsule, whereby the distal end of the capsule is compressible.

Example 12 relates to the device according to Example 1, wherein the capsule further comprises a void defined in a portion of the capsule, whereby an area of the capsule near the void is compressible.

In Example 13, a catheter advancement assistance device comprises a push rod, and a body fixedly attached to a distal end of the push rod. The body comprises a distal plug portion, a proximal neck portion, wherein the proximal neck portion has a smaller diameter than the distal plug portion, and a guidewire lumen defined through the body.

Example 14 relates to the device according to Example 13, wherein the guidewire lumen is sized to allow fluid to flow through the lumen when a standard guidewire is positioned therein.

Example 15 relates to the device according to Example 13, wherein the body further comprises a channel defined longitudinally along an outer surface of the body.

Example 16 relates to the device according to Example 13, wherein the body further comprises a seating component formed around at least a portion of an outer circumference of the capsule.

Example 17 relates to the device according to Example 16, wherein the seating component comprises a lip or a ridge.

In Example 18, a method of assisting advancement of a catheter through a blood vessel comprises inserting an advancement assistance device into a lumen of the catheter, urging the advancement assistance device distally into the lumen of the catheter until a distal portion of the distal plug portion extends out of a distal opening in the catheter and a proximal portion of the distal plug portion is positioned within the lumen of the catheter, urging the catheter distally into the blood vessel to a target site, and retracting the advancement assistance device from the catheter. The advancement assistance device comprises an elongate shaft and a body fixedly attached to a distal end of the elongate shaft. The body comprises a distal plug portion, a proximal neck portion, wherein the proximal neck portion has a smaller diameter than the distal plug portion, and a guidewire lumen defined through the body.

Example 19 relates to the method according to Example 18, further comprising urging the advancement assistance device distally until the distal plug portion extends out of the distal opening, whereby space is provided between the body and the distal opening, urging contrast solution distally through the catheter and through the space between the body and the distal opening and into the blood vessel, and urging the advancement assistance device proximally until the distal portion of the distal plug portion extends out of the distal opening in the catheter and the proximal portion of the distal plug portion is positioned within the lumen of the catheter.

Example 20 relates to the method according to Example 18, further comprising locking the advancement assistance device to the catheter after urging the device distally until the distal portion of the distal plug portion extends out of the distal opening in the catheter and the proximal portion of the distal plug portion is positioned within the lumen.

In Example 21, a method of assisting advancement of a catheter through a blood vessel comprises inserting an advancement assistance device into a lumen of the catheter, urging the advancement assistance device distally into the lumen of the catheter until a distal portion of the body extends out of a distal opening in the catheter and a proximal portion of the body is positioned within the lumen of the catheter, urging the catheter distally into the blood vessel to a target site, and retracting the advancement assistance device from the catheter. The advancement assistance device comprises an elongate shaft, and a body fixedly attached to a distal end of the elongate shaft, the body comprising a guidewire lumen defined through the body.

Example 22 relates to the method according to Example 21, further comprising locking the advancement assistance device to the catheter after urging the device distally until the distal portion of the body extends out of the distal opening in the catheter and the proximal portion of the body is positioned within the lumen.

Example 23 relates to the method according to Example 21, further comprising urging contrast solution distally through the catheter and through the guidewire lumen and into the blood vessel.

Example 24 relates to the method according to Example 21, wherein the body further comprises a channel defined longitudinally along an outer surface of the body.

Example 25 relates to the method according to Example 24, further comprising urging contrast solution distally through the catheter and through the channel and into the blood vessel.

Example 26 relates to the method according to Example 21, wherein the body further comprises a seating component formed around at least a portion of an outer circumference of the body.

Example 27 relates to the method according to Example 26, further wherein the seating component comprises a lip or a ridge.

Example 28 relates to the method according to Example 26, further comprising urging the advancement assistance device distally through the lumen of the catheter until the seating component is urged out of the distal opening in the catheter, and urging the advancement assistance device proximally until the seating component contacts the distal end of the catheter.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a cross-sectional view of a known catheter being advanced through a blooed vessel.

FIG. 1B is a cross-sectional view of a known balloon catheter positioned within a known catheter.

FIG. 2 is a cross-sectional side view of a catheter advancement device positioned within a catheter, according to one embodiment.

FIG. 3A is a cross-sectional side view of a distal portion of a plug of a catheter advancement device, according to one embodiment.

FIG. 3B is a cross-sectional side view of a distal portion of a plug of a catheter advancement device, according to another embodiment.

FIG. 4 is a cross-sectional side view of a catheter advancement device having visualization markers and positioned within a catheter, according to one embodiment.

FIG. 5 is a cross-sectional side view of a catheter advancement device extending out of a distal end of a catheter, according to one embodiment.

FIG. 6 is a cross-sectional side view of a catheter advancement device positioned within a catheter, according to another embodiment.

FIG. 7A is a cross-sectional side view of a catheter advancement device positioned within a catheter, according to a further embodiment.

FIG. 7B is a cross-sectional front view of the catheter advancement device of FIG. 7A.

FIG. 8 is a cross-sectional side view of a catheter advancement device positioned within a catheter, according to yet another embodiment.

FIG. 9 is a cross-sectional side view of a catheter advancement device positioned within a catheter, according to an alternative embodiment.

FIG. 10A is a cross-sectional side view of a plug, according to another alternative embodiment.

FIG. 10B is a cross-sectional front view of the plug of FIG. 10A.

FIG. 11A is a cross-sectional side view of a plug, according to another alternative embodiment.

FIG. 11B is a further cross-section side view of the plug of FIG. 11A.

FIG. 12 is a cross-sectional side view of a method of making a catheter advancement device, according to one embodiment.

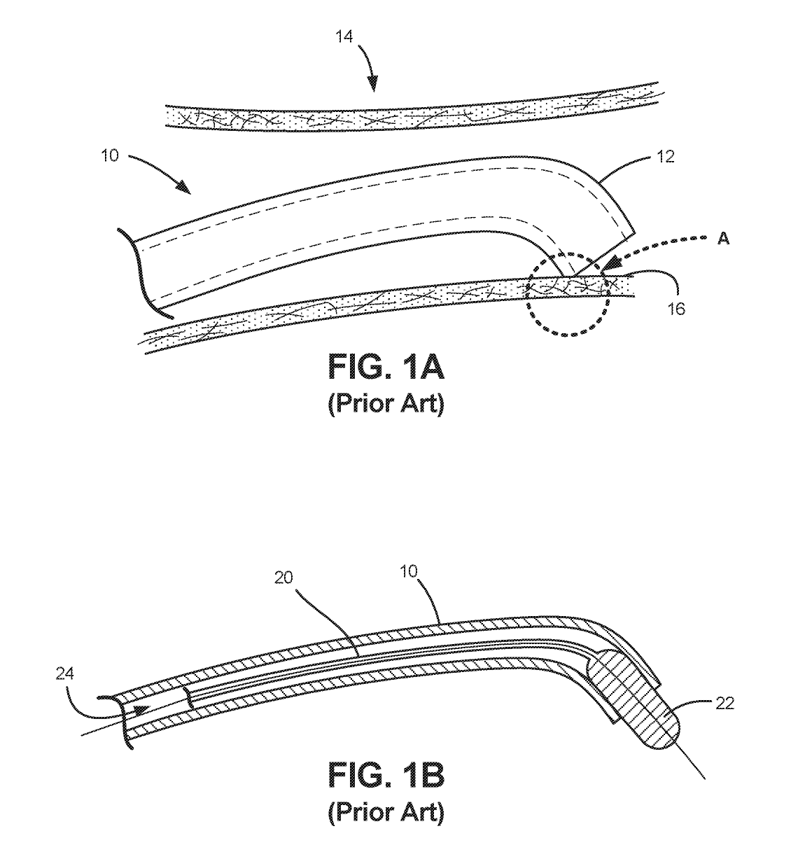

### DETAILED DESCRIPTION

The various embodiments disclosed and contemplated herein relate to methods and devices for assisting in the advancement of a catheter, including, for example, a guiding catheter with a curved shape, while reducing or eliminating the risk of damage to the blood vessel inner wall. Alternatively, the various methods and devices disclosed or contemplated herein can be used to assist in the advancement of any type of catheter, pre-shaped or otherwise. It is understood that while many of the exemplary embodiments disclosed herein discuss guiding catheters, the various device implementations disclosed or contemplated herein can be used with any guiding, delivery, or other type of catheter or sheath.

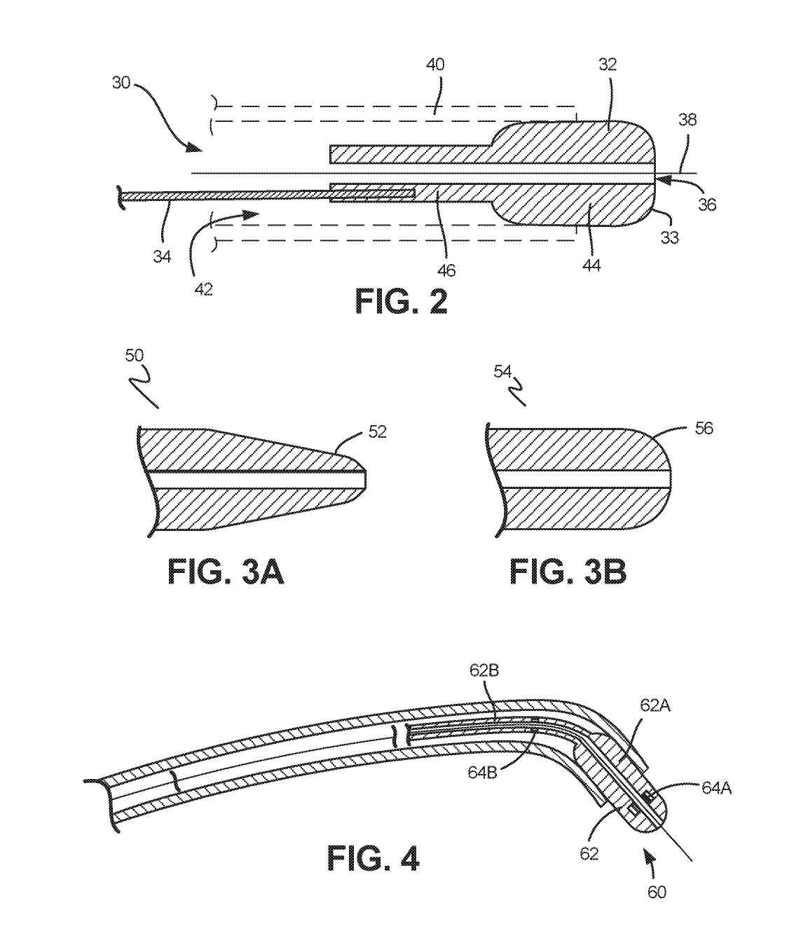

FIG. 2 depicts, according to one embodiment, a catheter advancement device 30 (also referred to as an “insertion device,” a “catheter insertion device,” or an “advancement device”) for assisting with or use in advancing a catheter, such as, for example, a guiding catheter. The device 30 has a capsule 32 with a partially rounded distal end 33 and a push rod (also referred to herein as an “elongate component”) 34 coupled thereto. The capsule 32 has a lumen 36 defined therethrough that is configured to allow for passage of a guidewire 38 therethrough as shown. The push rod 34 is embedded in or otherwise fixedly coupled to the capsule 32 such that appropriate forces can be applied at the proximal end of the push rod 34 by a user (such as a surgeon or medical professional) to urge the capsule 32 distally or proximally during use.

In use as shown in the figure, the capsule insertion device 30 can be advanced through the inner lumen 42 of a guiding catheter 40 (by a user holding the proximal end of the rod 34) and positioned such that the capsule 32 is protruding from the distal end of the catheter 40. In this position, a portion of the capsule 32 is positioned within the distal end of the catheter 40 and a portion extends out of the distal end of the catheter 40. At this point, the user locks the device 30 into position in relation to the catheter 40. That is, according to one embodiment, the user attaches the proximal end of the rod 34 to the catheter 40 in any known fashion. For example, in one specific implementation, a known locking mechanism at the proximal end of the catheter 40 (such as a Tuohy-Borst adapter, for example) is used to lock the device 30 to the catheter 40. As such, the device 30 is locked or otherwise attached to the catheter 40 such that the device cannot move translationally in relation to the catheter 40. Once the capsule 32 is positioned as shown (and, in some cases, the device 30 is locked in place), the guiding catheter 40 can be advanced through a blood vessel with the capsule 32 positioned to prevent direct contact between the distal end of the catheter 40 and the inner wall of the vessel, thereby preventing the “razor effect.” Once the catheter 40 is advanced to the desired position, the capsule catheter 30 is withdrawn from the guiding catheter 40 by a user pulling the push rod 34 in the proximal direction so that the guiding catheter 40 is ready for use.

In this specific implementation, the capsule 32 has a distal portion (also referred to herein as a “plug,” “distal plug,” or “distal body”) 44 and a proximal portion (also referred to as a “neck,” or “tail”) 46. In certain implementations such as that depicted in FIG. 2, the distal body 44 has a larger diameter than the neck 46.

Alternatively, in the various embodiments disclosed or contemplated herein, the capsule (such as capsule 32) can be any component or body that can be inserted through a guiding catheter and positioned to reduce or eliminate the razor effect. According to some embodiments, the capsule is non-inflatable. In certain implementations, the capsule has a substantially cylindrical shape. Alternatively, the capsule can have any known shape that allows it to be advanced through a catheter and positioned out of the distal end thereof as described herein.

In one embodiment, any capsule embodiment disclosed or contemplated herein (such as capsule 32) is made of a polymeric material. For example, the capsule can be made of polyethylene, Pebax, Nylon, polyester, or any other polymeric material or combination thereof. Alternatively, the capsule can also be made of metal or any other known material that can be used for medical devices. In a further implementation, the capsule (such as capsule 32) can be made of two or more materials. More specifically, in certain embodiments, the capsule can be made of two or more materials having differing stiffness and/or flexibility such that one portion of the capsule (such as the distal end, for example) is stiffer, more rigid, and/or less flexible than another portion. In other words, the two or more materials can be used to create a capsule that has stiffness, rigidity, or flexibility that varies along the length of the capsule.

According to any embodiment disclosed or contemplated herein, the push rod (such as push rod 34) can be made of metal. Alternatively, the push rod can be made of any known material that can be used to make a substantially stiff or inflexible component that can be used to advance a capsule through the lumen of a guiding catheter. It is understood that the push rod (also referred to as a “proximal elongate member” or “control rod” or “manipulation rod”) in any embodiment herein can be any elongate component that is coupled to the proximal end of the capsule and can withstand the forces necessary for a user to urge the rod distally or proximally to move the capsule through a guiding catheter as described herein. In certain alternative implementations, the push rod can be integral with the capsule. Further, various embodiments include a push rod and capsule formed together of the same materials such that the push rod is integral with the capsule.

It is understood that the capsule (such as capsule 32) can take a variety of shapes, so long as the capsule can be positioned out of the distal end of a guiding catheter as described herein and help with advancement thereof through a blood vessel. For example, capsule 50 as best shown in FIG. 3A according to one embodiment has a tapered distal end 52, while capsule 54 as best shown in FIG. 3B in accordance with another implementation has a rounded distal end 56. According to further alternatives, the capsule can have a distal end with an angled shape, a spherical shape, or any other known shape that helps to advance the guiding catheter when the capsule is positioned out of the distal end of the guiding catheter.

In certain implementations, any advancement device disclosed or contemplated herein can have at least one visualization marker disposed on the device. One exemplary embodiment is depicted in FIG. 4, in which the capsule insertion device 60 has two visualization markers 64A, 64B. The first visualization marker 64A is disposed at or near the distal end of the distal plug 62A of the capsule 62, while the second marker 64B is disposed on the neck 62B of the capsule 62. Alternatively, any device embodiment can have one marker or three or more markers. It is understood that the markers 64A, 64B (and any markers incorporated into any capsule insertion device embodiment as disclosed or contemplated herein) can be radiopaque markers. Alternatively, the markers (such as markers 64A, 64B) can be made of any known material for a visualization marker. The markers 64A, 64B—and any such markers used in any embodiment herein—can be used to assist a user with positioning the capsule insertion device 60.

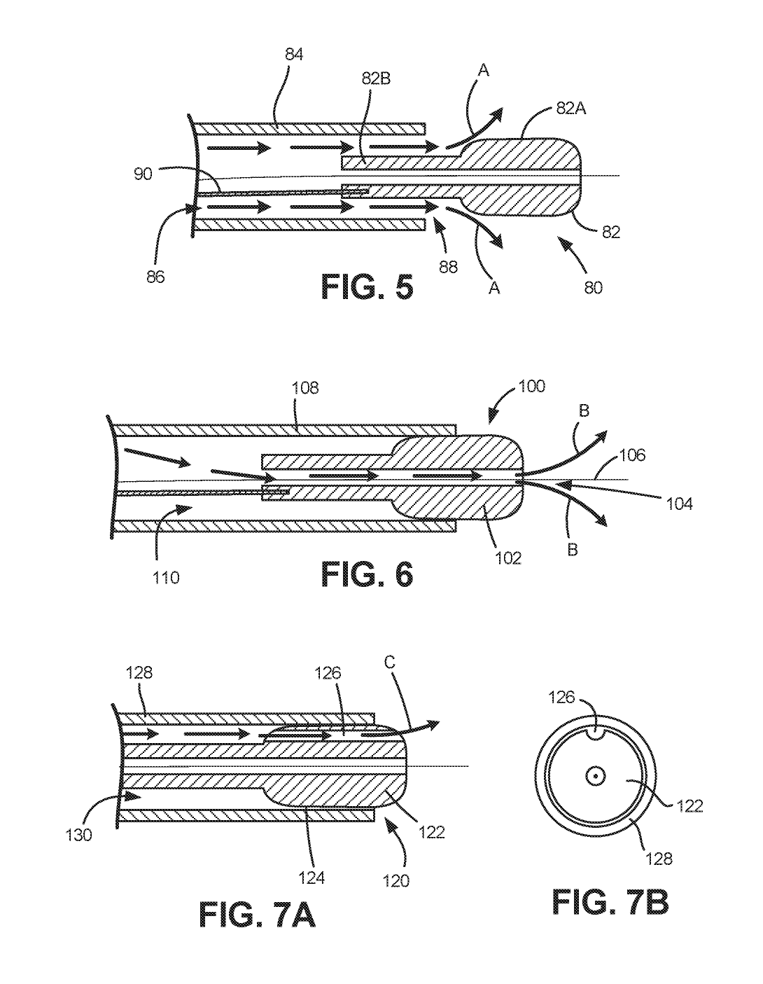

In use, it is often necessary or helpful to inject contrast solution through the lumen of a guiding catheter and into the vasculature of the patient to assist with placement of the guiding catheter. As discussed above, if a known balloon catheter is being used to assist with advancement of the guiding catheter, the process for injecting the contrast solution is complicated by the presence of the balloon, which must be deflated in order to inject the solution. However, as best shown in FIGS. 5-7B, various embodiments of the capsule device disclosed or contemplated herein eliminate those complications.

For example, in one implementation as shown in FIG. 5, the catheter advancement device 80 has a capsule 82 with a distal body 82A and a neck 82B. To inject contrast solution after the capsule 82 has been positioned at the distal end of the guiding catheter 84 (for advancing the catheter 84 through the vasculature as described above), the capsule device 80 is urged distally (by a user urging the proximal end of the push rod 90 distally) such that the body 82A of the capsule 82 is urged distally out of the lumen 86 of the guiding catheter 84 as shown in FIG. 5. More specifically, the capsule 82 is urged distally until the body 82A is urged out of the lumen 86 such that space is created between the opening 88 of the guiding catheter 84 and the body 82A, thereby making it possible for contrast to exit from the opening 88 as represented by arrows A. In certain implementations such as that shown in FIG. 5, the capsule 82 need not be urged distally so far that the neck 82B also exits the lumen 86. Instead, the smaller diameter of the neck 82B allows for sufficient space between the neck 82B and the opening 88 to allow for contrast to exit the lumen 86.

In an alternative embodiment, as shown in FIG. 6, the capsule 102 need not be advanced distally out of the lumen 110 of the guiding catheter 108. More specifically, one implementation of a capsule device 100 has a capsule 102 with a lumen 104 defined therein that is larger than is necessary to accommodate solely a guide wire 106. As such, the lumen 104 has a sufficient inner diameter to provide space for the guide wire 106 while also having sufficient additional space to allow for contrast solution to flow distally out of the guiding catheter 108 through the capsule lumen 104 and into the vasculature as represented by arrows B. In use, once the capsule device 100 is positioned at the distal end of the guiding catheter 108 such that the capsule 102 is positioned as desired for advancing the catheter 108, the capsule 102 need not be moved in order to inject the contrast solution. Instead, the capsule 102 can remain in place while the contrast solution flows distally through the lumen 110 of the guiding catheter 108 and through the lumen 104 of the capsule 102 and out into the vasculature.

Another configuration as depicted in FIGS. 7A and 7B also allows for contrast solution injection without moving the capsule device. In this implementation, the capsule device 120 has a capsule 122 with a channel (also referred to as a “slot” or “trough”) 126 defined longitudinally along the outer surface 124 of the capsule 122. As such, the channel 126 defines a space between the capsule 122 and the inner surface of the lumen 130 of the guiding catheter 128 through which contrast solution can flow distally out into the vasculature as represented by arrows C. In use, once the capsule device 120 is positioned at the distal end of the guiding catheter 128 such that the capsule 122 is positioned as desired for advancing the catheter 128, the capsule 122 need not be moved in order to inject the contrast solution. Instead, the capsule 122 can remain in place while the contrast solution flows distally through the lumen 130 of the guiding catheter 128 and through the channel 126 of the capsule 122 and out into the vasculature. It is understood that, according to various alternatives, the channel 126 can be any feature or configuration on the capsule 122 or the outer surface 124 thereof that allows fluid flow between the capsule 122 and the inner wall of the lumen 130.

Both the larger lumen 104 of the capsule device 100 embodiment and the channel 126 of the capsule device 120 implementation make it easy for a user to inject contrast solution, as discussed above. In addition, these two embodiments can also assist with limiting contrast fluid use. Injection of excess contrast fluid into the vasculature of a patient during an interventional procedure can cause health issues for the patient, including contrast-induced nephropathy. The lumen 104 of a predetermined diameter in the device 100 embodiment or the channel 126 of a predetermined depth or diameter in the device 120 embodiment both provide mechanisms for injecting solution in known, more limited amounts than those injected when using a balloon catheter as described above.

In certain alternative implementations, a capsule is provided—for use with any of the capsule device embodiments disclosed or contemplated herein—that has a seating component defined or disposed around an outer surface of the capsule that can assist with positioning the capsule in relation to the guiding catheter during use and further can create a smoother or more streamlined transition from the outer surface of the capsule to the outer surface of the guiding catheter in which the capsule is positioned. It is understood that any of the seating components disclosed or contemplated herein can be defined or disposed around the entire 360 degree circumference of the capsule. Alternatively, any such components can be defined or disposed around only a portion of the circumference of the capsule. In a further embodiment, any such seating component can be defined or disposed intermittently around the circumference of the capsule such that there are two or more seating components disposed or defined thereon such that they are positioned at different locations along and around the circumference thereof.

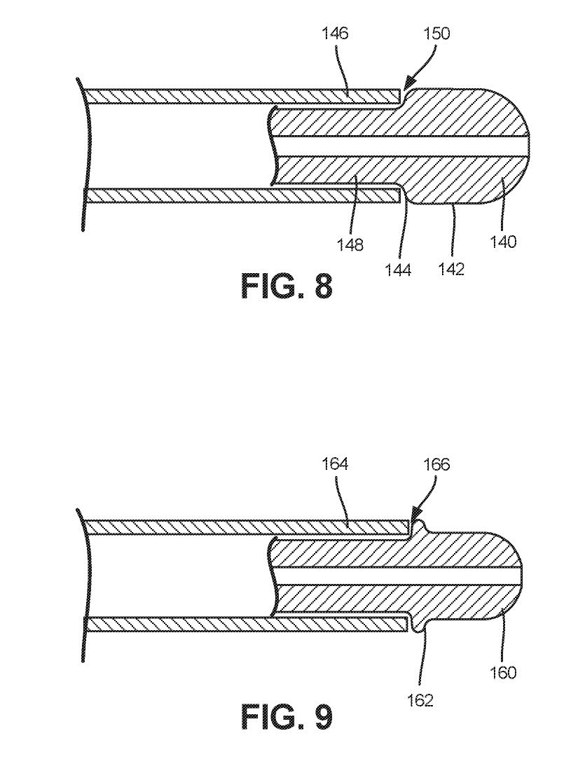

One example of such a capsule is depicted in FIG. 8, which shows one embodiment of a capsule 140 having a seating component (or “seating feature”) 144. In this specific example, the seating component 144 is a lip 144 created by the capsule 140 having an expanded section 142 (which is a portion of the capsule 140 that has an increased diameter in comparison to the rest of the capsule 140). That is, the lip 144 is formed at the juncture of the expanded section 142 and the non-expanded section of the capsule 140. As discussed above, the lip 144 can extend around the entire circumference, can extend around a portion of the circumference, or can constitute two or more lips 144 that are disposed or defined intermittently around the circumference. In one embodiment, the expanded section 142 is a portion of the body 148 of the capsule 140. Alternatively, the expanded section 142 can be the body itself (not shown), and the transition from the body (not shown) to the neck (not shown) constitutes the seating component. The expanded section 142 has an outer diameter that is substantially similar to or the same as the outer diameter of the guiding catheter 146. In certain embodiments, the expanded section 142 or the entire capsule 140 has sufficient elasticity to allow for deformation of the expanded section 142 such that the capsule 140 can be advanced through the guiding catheter 146 despite the expanded section 142 having an outer diameter that is larger than the inner diameter of the guiding catheter 146 lumen.

In use, the capsule 140 is advanced distally through the guiding catheter 146 and positioned out of the distal opening 150 of the guiding catheter 146 according the same procedure used for all the capsule device embodiments herein. As mentioned above, the capsule 140 has an expanded section 142 that has elastic characteristics that allow for the section 142 to deform sufficiently as the capsule 140 is advanced through the guiding catheter to allow for passage of the capsule 140 despite the expanded section 142 having a greater diameter than the inner diameter of the lumen of the guiding catheter 146. As the expanded section 142 of the capsule 140 is urged out of the opening 150 at the distal end of the guiding catheter 146, the expanded section 142 expands back to its natural diameter, thereby causing formation of the lip 114. The user can then urge the capsule 140 back in a proximal direction—via the push rod (not shown)—until the lip 114 is in contact with the guiding catheter 146, thereby confirming for the user via increased resistance that the capsule 140 is in the desired position in relation to the guiding catheter 146. It is understood that the user must be aware that she or he cannot use so much force that the expanded section 142 deforms and the capsule 140 is urged proximally past the desired capsule 140 position. Once the capsule 140 is positioned as desired, it can be seen in FIG. 8 that the expanded section 142 has a diameter that is substantially similar to the outer diameter of the guiding catheter 146, thereby reducing the risk of the distal end of the guiding catheter 146 making contact with an inner wall of a blood vessel wall during advancement of the guiding catheter 146. Once the capsule 140 is positioned, the user then advances the guiding catheter 146 via the push rod (not shown).

Another example of a capsule with a seating component is depicted in FIG. 9, which shows a capsule 160 having a seating component 162. In this specific example, the seating component 162 is a ridge 162 formed or disposed on the outer surface of the capsule 160. As discussed above, the ridge 162 can extend around the entire circumference, can extend around a portion of the circumference, or can constitute two or more ridges 162 that are disposed or defined intermittently around the circumference. The ridge 162 has an outer diameter that is substantially similar to or the same as the outer diameter of the guiding catheter 164. In certain embodiments, the ridge 162 or the entire capsule 160 has sufficient elasticity to allow for deformation of the ridge 162 such that the capsule 160 can be advanced through the guiding catheter 164 despite the ridge 162 having an outer diameter that is larger than the inner diameter of the guiding catheter 164 lumen.

In use, the capsule 160 is advanced distally through the guiding catheter 164 and positioned out of the distal opening 166 of the guiding catheter 164 according the same procedure used for all the capsule device embodiments herein. As mentioned above, the ridge 162 on the capsule 160 has elastic characteristics that allow for the ridge 162 to deform sufficiently as the capsule 160 is advanced through the guiding catheter 164 to allow for passage of the capsule 160 despite the ridge 162 having a greater diameter than the inner diameter of the lumen of the guiding catheter 164. As the ridge 162 of the capsule 160 is urged out of the opening 166 at the distal end of the guiding catheter 164, the ridge 162 expands back to its natural diameter. The user can then urge the capsule 160 back in a proximal direction until the ridge 162 is in contact with the guiding catheter 164, thereby confirming that the capsule 160 is in the desired position in relation to the guiding catheter 164. It is understood that the user must be aware that the user cannot use so much force that the ridge 162 deforms and the capsule 160 is urged proximally past the desired capsule 160 position. Once the capsule 160 is positioned as desired, it can be seen in FIG. 9 that the ridge 162 has a diameter that is substantially similar to the outer diameter of the guiding catheter 164, thereby reducing the risk of the distal end of the guiding catheter 164 making contact with an inner wall of a blood vessel wall during advancement of the guiding catheter 164. Once the capsule 160 is positioned, the user then advances the guiding catheter 164.

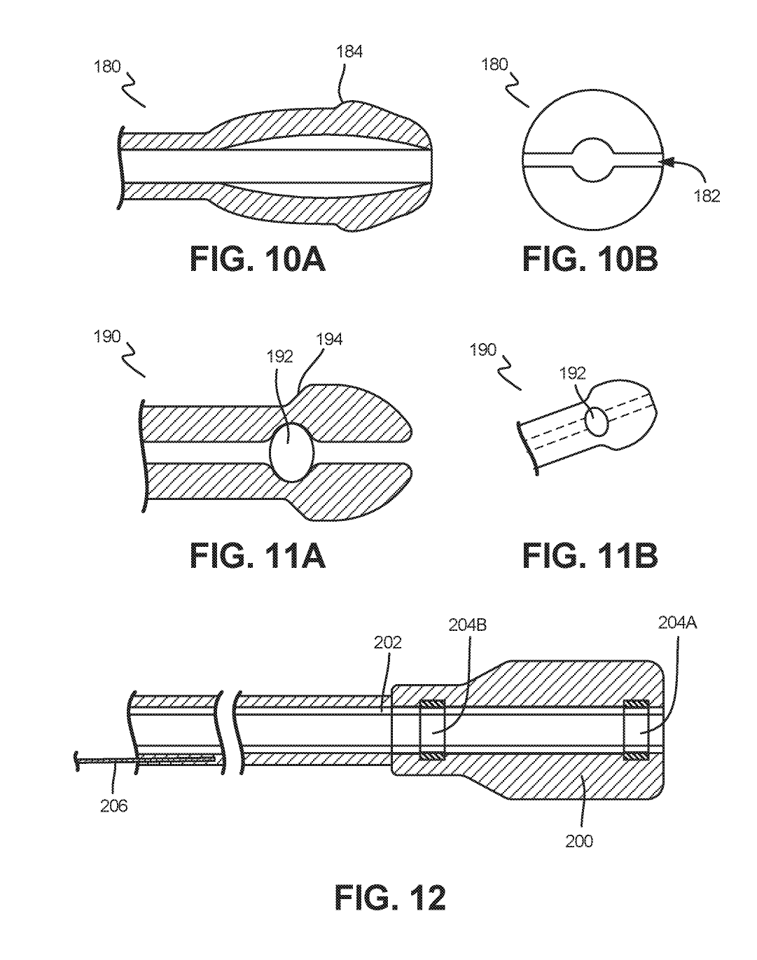

In certain implementations in which the capsule has a seating component (such as the seating components 144, 162 described above, for example) or similar feature, the deformation or partial collapse of the capsule makes it possible for the capsule to advance through the guiding catheter as discussed above. In one exemplary embodiment as shown in FIGS. 10A and 10B, instead of the capsule having elasticity as described above, a capsule 180 is provided that is a deformable or collapsible capsule 180. That is, the capsule 180 has a slot 182 defined in the distal end of the capsule 180 along the length of the capsule 180 that also has a seating component 184 (similar to one of the seating components 144, 162 described above). In use, the slot 182 allows for the capsule 180 to be deformed or have a smaller diameter as it is advanced through a guiding catheter, similar to the use of the capsules 140, 160 discussed above.

In another embodiment, a capsule 190 is provided that is collapsible or deformable as a result of an opening 192 defined at a distal portion of the capsule along the length of the capsule 190 that also has a seating component 194. In use, the opening 192 allows for the capsule 190 to be deformed or have a smaller diameter as it is advanced through a guiding catheter, similar to the use of the capsules 140, 160 discussed above.

As discussed above, it is understood that the seating components 184, 194 described above with respect to FIGS. 10A-11B can extend around the entire circumference, can extend around a portion of the circumference, or can constitute two or more such components that are disposed or defined intermittently around the circumference.

The various catheter insertion device embodiments disclosed or contemplated herein can be made in any number of known ways. In one embodiment as shown in FIG. 12, a capsule 200 can be formed using an injection molding process. In this process, the starting point is an inner tube 202, with the capsule 200 being injection molded over the inner tube 202. In certain implementations in which the capsule 200 has two marker bands 204A, 204B, the marker bands 204A, 204B are disposed over the inner tube 202 before the capsule 200 is injected molded thereon such that the material for the capsule 200 is injection molded onto the marker bands 204A, 204B, thereby resulting in the marker bands 204A, 204B being embedded in the capsule 200. Further, the push rod 206 can be embedded in the capsule 200 in a similar fashion. That is, the push rod 206 can be positioned along the inner tube 202 such that injection molding of the capsule 200 results in the push rod 206 being embedded therein.

Although the present invention has been described with reference to preferred embodiments, persons skilled in the art will recognize that changes may be made in form and detail without departing from the spirit and scope of the invention.

What is claimed is:

1. A catheter advancement assistance device, the device comprising:
(a) an elongate shaft; and
(b) a capsule fixedly attached to a distal end of the elongate shaft, the capsule comprising:
(i) a guidewire lumen defined through the capsule;
(ii) an outer diameter substantially similar to an inner diameter of a catheter such that the capsule is sized to be positionable through the catheter.

2. The device of claim 1, wherein the capsule further comprises:
(a) a distal portion; and
(b) a neck extending proximally from the distal portion, wherein the neck has a smaller diameter than the distal portion.

3. The device of claim 1, wherein the guidewire lumen has an inner diameter that is larger than an outer diameter of a standard guidewire.

4. The device of claim 1, wherein the guidewire lumen is sized to allow fluid to flow through the lumen when a standard guidewire is positioned therein.

5. The device of claim 1, wherein the capsule further comprises a channel defined longitudinally along an outer surface of the capsule.

6. The device of claim 1, wherein the capsule further comprises a lip formed around at least a portion of an outer circumference of the capsule.

7. The device of claim 6, wherein the lip comprises at least two lip segments formed around the outer circumference of the capsule.

8. The device of claim 1, wherein the capsule further comprises:
(a) an expanded distal section, wherein the expanded distal segment is substantially elastic;
(b) a non-expanded proximal section having a smaller diameter than the expanded distal section; and
(c) a lip formed at a juncture between the expanded distal section and the non-expanded proximal section, wherein the lip is formed around at least a portion of a circumference of the capsule.

9. The device of claim 1, wherein the capsule further comprises a substantially elastic ridge formed around at least a portion of an outer circumference of the capsule.

10. The device of claim 9, wherein the substantially elastic ridge comprises at least two substantially elastic rig segments formed around the outer circumference of the capsule.

11. The device of claim 1, wherein the capsule further comprises a slot defined in a distal end of the capsule, whereby the distal end of the capsule is compressible.

12. The device of claim 1, wherein the capsule further comprises a void defined in a portion of the capsule, whereby an area of the capsule near the void is compressible.

13. A catheter advancement assistance device, the device comprising:
(a) a push rod; and
(b) a body fixedly attached to a distal end of the push rod, the body comprising:
(i) a distal plug portion;
(ii) a proximal neck portion, wherein the proximal neck portion has a smaller diameter than the distal plug portion; and
(iii) a guidewire lumen defined through the body.

14. The device of claim 13, wherein the guidewire lumen is sized to allow fluid to flow through the lumen when a standard guidewire is positioned therein.

15. The device of claim 13, wherein the body further comprises a channel defined longitudinally along an outer surface of the body.

16. The device of claim 13, wherein the body further comprises a seating component formed around at least a portion of an outer circumference of the capsule.

17. The device of claim 16, wherein the seating component comprises a lip or a ridge.

18. A method of assisting advancement of a catheter through a blood vessel, the method comprising:
inserting an advancement assistance device into a lumen of the catheter, the advancement assistance device comprising:
(a) an elongate shaft;
(b) a body fixedly attached to a distal end of the elongate shaft, the body comprising:
(i) a distal plug portion;
(ii) a proximal neck portion, wherein the proximal neck portion has a smaller diameter than the distal plug portion; and
(iii) a guidewire lumen defined through the body;
urging the advancement assistance device distally into the lumen of the catheter until a distal portion of the distal plug portion extends out of a distal opening in the catheter and a proximal portion of the distal plug portion is positioned within the lumen of the catheter;
urging the catheter distally into the blood vessel to a target site; and
retracting the advancement assistance device from the catheter.

19. The method of claim 18, further comprising:
urging the advancement assistance device distally until the distal plug portion extends out of the distal opening, whereby space is provided between the body and the distal opening;
urging contrast solution distally through the catheter and through the space between the body and the distal opening and into the blood vessel; and
urging the advancement assistance device proximally until the distal portion of the distal plug portion extends out of the distal opening in the catheter and the proximal portion of the distal plug portion is positioned within the lumen of the catheter.

20. The method of claim 18, further comprising locking the advancement assistance device to the catheter after urging the device distally until the distal portion of the distal plug portion extends out of the distal opening in the catheter and the proximal portion of the distal plug portion is positioned within the lumen.

21. A method of assisting advancement of a catheter through a blood vessel, the method comprising:
inserting an advancement assistance device into a lumen of the catheter, the advancement assistance device comprising:
(a) an elongate shaft;
(b) a body fixedly attached to a distal end of the elongate shaft, the body comprising a guidewire lumen defined through the body;
urging the advancement assistance device distally into the lumen of the catheter until a distal portion of the body extends out of a distal opening in the catheter and a proximal portion of the body is positioned within the lumen of the catheter;
urging the catheter distally into the blood vessel to a target site; and
retracting the advancement assistance device from the catheter.

22. The method of claim 21, further comprising locking the advancement assistance device to the catheter after urging the device distally until the distal portion of the body extends out of the distal opening in the catheter and the proximal portion of the body is positioned within the lumen.

23. The method of claim 21, further comprising urging contrast solution distally through the catheter and through the guidewire lumen and into the blood vessel.

24. The method of claim 21, wherein the body further comprises a channel defined longitudinally along an outer surface of the body.

25. The method of claim 24, further comprising urging contrast solution distally through the catheter and through the channel and into the blood vessel.

26. The method of claim 21, wherein the body further comprises a seating component formed around at least a portion of an outer circumference of the body.

27. The method of claim 26, wherein the seating component comprises a lip or a ridge.

28. The method of claim 26, further comprising:
urging the advancement assistance device distally through the lumen of the catheter until the seating component is urged out of the distal opening in the catheter; and
urging the advancement assistance device proximally until the seating component contacts the distal end of the catheter.
